(19) United States
(12) Patent Application Publication
(10) Pub. No.: US 2014/0180077 A1
(43) Pub. Date: Jun. 26, 2014

(54) TISSUE ABLATION CATHETER AND METHODS OF ABLATING TISSUE

(71) Applicant: VOLCANO CORPORATION, San Diego, CA (US)

(72) Inventors: Scott Huennekens, San Diego, CA (US); Cheryl D. Rice, San Diego, CA (US)

(73) Assignee: VOLCANO CORPORATION, San Diego, CA (US)

(21) Appl. No.: 14/133,226

(22) Filed: Dec. 18, 2013

Related U.S. Application Data
(60) Provisional application No. 61/745,248, filed on Dec. 21, 2012.

(51) Int. Cl. A61B 18/08; A61B 8/00; A61B 5/00; A61B 18/14

(52) U.S. Cl. CPC A61B 18/082; A61B 18/1492; A61B 8/445; A61B 5/0073
USPC 600/425; 606/31; 600/407; 600/470

(57) ABSTRACT

Catheters having expandable members, e.g., balloons, incorporating heating elements and temperature sensors for controlled delivering of energy to tissues, i.e., to treat diseases, especially hypertension. The invention also describes methods for monitoring and controlling the amount of energy delivered to the tissue.

### RELATED APPLICATIONS

This application claims priority to U.S. Provisional Application No. 61/745,248, filed Dec. 21, 2012, which is incorporated herein by reference in its entirety.

### FIELD OF THE INVENTION

The invention relates to catheters having expandable members, e.g., balloons, for delivering energy to tissues, i.e., to treat the tissues. The invention also describes methods for monitoring and controlling an amount of energy delivered to the tissue.

### BACKGROUND

Physicians use catheters to gain access to and repair interior tissues of the body, particularly within the lumens of the body such as blood vessels. For example, balloon angioplasty and other catheters often are used to open arteries that have been narrowed due to atherosclerotic disease. Catheters can also be used to deliver devices, e.g., stents or valves to the vasculature. Another common catheter use is to deliver therapy to a tissue, such as a drug, heat, or other forms of energy.

The process of heating a tissue to treat a disorder is generally known as “ablation,” even when the tissue is not removed. When ablation techniques were first pioneered, they were truly ablative, in that layers of tissue were burned away with high temperature tools. It has since been discovered that many disorders can be treated by merely heating, but not necessarily removing the tissue, because the heating causes changes to the tissue, e.g., scarring, or destroys/diminishes vasculature or nerves underlying the tissue. For example, endometrial ablation is commonly used to control uterine bleeding. Endometrial ablation involves heating the tissue of the uterine lining to cause the tissue to scar and to dilate the underlying vasculature.

A newer ablation procedure, known as renal denervation (RDN), uses ablative techniques to damage nerves in the walls of the renal arteries. Damage to the nerves in this area affects sympathetic drive, a part of the autonomic nervous system that controls certain body functions when the body is exposed to stress. In particular, destruction of the nerves adjacent to the renal artery results in lower blood pressure, partially because the mechanism by which blood pressure is elevated due to stress is muted. Where approved, the procedure can be used to treat patients that do not respond sufficiently to hypertension medication. Early clinical trials have shown that patients undergoing this procedure commonly experience a sustained decrease in systolic blood pressure of 25-32 mmHg, and a sustained decrease in diastolic pressure of 12-18 mmHg. See “Symplicity™ RDN System Clinical Trial Data,” at http://www.medtronicrdn.com/intl/healthcare-professionals/symplicity-rdn-system/symplicity-clinical-trial-data/index.htm.

Accordingly, there is a need for advanced ablation devices for performing procedures such as renal denervation.

### SUMMARY

The invention provides methods and devices for heating an inflation fluid in an ablation balloon catheter in order to improve the accuracy of sensors in the balloon. For example, in order to reduce measurement errors, an ablation balloon is filled with an inflation fluid having substantially the same temperature as the target tissue treatment temperature. Because the inflation fluid is heated, the sensors contacting the tissue experience less convective cooling, and the accuracy of tissue temperature measurement is improved. Thus, using the methods and devices of the invention, a surgeon can be assured that the treated tissues were not overheated, but were raised to a temperature sufficient to affect the desired outcome. The treatment methods of the invention are broadly applicable to any ablative procedure, i.e., wherein energy (e.g., heat) is provided to a tissue to affect a therapeutic change.

Additionally, as described in detail below, the invention includes an expandable balloon having a first temperature sensor integrated into a balloon wall and a second temperature sensor at the distal end of the catheter, typically located away from the balloon wall. The two temperature sensors allow independent measurements of the temperature of the tissue being treated and the temperature of a heated fluid, respectively. The disclosed design increases the accuracy of the tissue temperature measurement made by the first temperature sensor, and results in better outcomes for ablative procedures. In some embodiments, the catheter additionally includes a heating element configured to heat the fluid within the balloon.

Using the disclosed ablation balloon and methods for applying energy to a subject, it will be safer to ablate tissues, and it will be easier to verify that the tissues have been properly heated to achieve the desired results.

### BRIEF DESCRIPTION OF THE DRAWINGS

FIG. 1 is a generalized depiction of a balloon catheter;

FIG. 2A shows delivery of a balloon catheter of the invention to a tissue in need of treatment;

FIG. 2B shows the balloon of FIG. 2A being inflated;

FIG. 2C shows the tissue being treated by increasing the temperature with RF energy;

FIG. 3 shows an embodiment of an ablation balloon for controlled delivery of energy to a tissue;

FIG. 4 shows an embodiment of an ablation balloon for controlled delivery of energy to a tissue;

FIG. 5 shows a flowchart describing an embodiment of a method for controlling delivery of energy to a tissue.

### DETAILED DESCRIPTION

The invention provides improved balloon catheters and methods of using the catheters, as well as other expandable devices, to deliver energy to tissues in need of treatment. In particular, the catheters of the invention allow active monitoring of tissue temperatures to reduce the rate of errors in delivering ablative treatment. Because the catheters of the invention use heated fluids in the balloon, there is less error in the temperature measurements due to convective heat loss. While the description focuses primarily on renal artery ablation for renal denervation (RDN) the devices and methods are broadly applicable to other ablative procedures, such as endometrial ablation or resculpting of atherosclerotic vessels, among others.

Ablation procedures typically involve contacting a tissue with a hot tool, such as a catheter, or fluid. The heating process often kills the outermost layer of cells contacting the object, and may damage or modify layers of cells below the outermost layer. Some ablation procedures use directed energy to heat and modify the outermost layer of cells, or a nearby layer of cells (treatment depth). In some embodiments, lasers, microwaves, or radiofrequency (RF) waves are directed at the tissue, causing the tissue to heat to treatment temperatures. Typically, the energy is absorbed directly, thus causing the tissue to heat. In some embodiments, a secondary structure, e.g., an antenna, receives the directed energy and heats the tissues. During a procedure the temperature of the tissue is typically elevated to 50° C. or greater, e.g., 55° C. or greater, e.g., 60° C. or greater, e.g., 65° C. or greater, e.g., 70° C. or greater. In some embodiments the tissue is heated to about 65° C., e.g., 68° C.

In order to minimize risks when performing ablative procedures such as renal denervation (RDN), it is important to monitor and control the temperature of the device and the surrounding tissues. For example, during RDN, the renal artery could be weakened, increasing the chance of embolism, or the renal artery could be perforated or severed. To avoid such damage, prior art devices rely on gated energy delivery to control the temperature of the tissue. That is, RDN devices are programmed to provide predetermined dosing times and wattage based upon accumulated experience and animal/cadaver studies. For example, 4 Watts of radiofrequency energy delivered for 2 seconds has been found to increase the temperature of a cadaver aorta to 65° C. with a particular balloon ablation device. See U.S. Patent Publication No. 2012/0158101 incorporated by reference herein in its entirety. Operation within the suggested range is assumed to provide safe and effective treatment. Nonetheless, without active temperature monitoring, it is impossible to know if the renal artery tissue is overheating. It is also difficult to assure that the target tissue was, in fact, raised to a temperature suitable to denerve the tissue. Using prior art methods, it is impossible to determine if the tissue has been adequately denerved without prolonged blood pressure monitoring after the procedure.

In order to address these concerns, the invention places an expandable member (e.g., a balloon) having a temperature sensor in proximity to the tissues (e.g., blood vessels), and expands the expandable member to cause the expandable member to contact the tissue and deliver therapy. For example, a heated fluid, having a temperature similar to a target therapy temperature, can be provided to the expandable member to increase the accuracy of tissue temperature measurements. During the ablative procedure, i.e., while energy is applied to the tissue via the expandable member, the tissue temperature is measured with a sensor in the presence of the heated fluid. Based upon the temperature assessments, the treatment is continued until the tissue has reached a target therapeutic temperature. Because the temperature measurement is more accurate than current methods, it is less likely that the tissue will be overheated, and it is more likely that the tissue will reach the target temperature during the procedure.

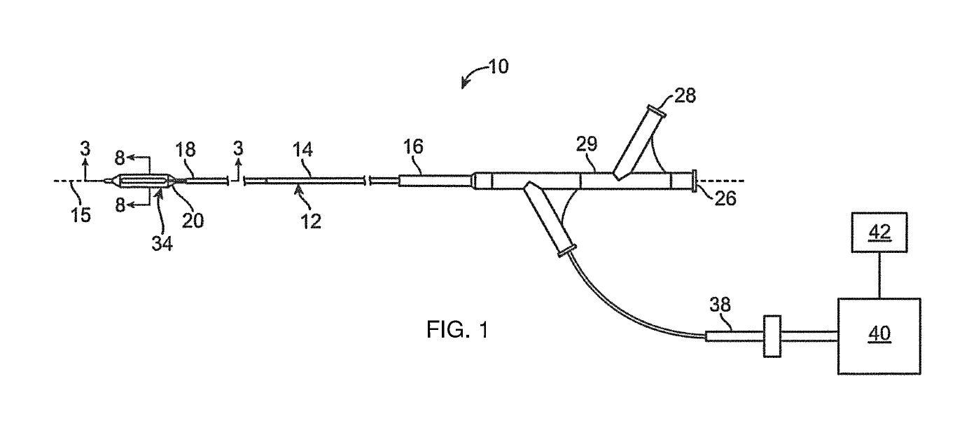

FIG. 1 shows an embodiment of a balloon ablation catheter system 10 for treating tissues with heat. The catheter system 10 includes a balloon catheter 12 having a catheter body 14 with a proximal end 16 and a distal end 18. Catheter body 14 is flexible and defines a catheter axis 15, and may include one or more lumens, such as a guide wire lumen and an inflation lumen. Additional lumens may be provided for other treatments, such as imaging, perfusion, fluid delivery, etc. Catheter 12 includes an inflatable balloon 20 adjacent distal end 18 and a housing 29 adjacent proximal end 16. When inflated and energized, inflatable balloon 20 provides thermal RF energy to the tissue, causing it to increase in temperature. Housing 29 includes a first connector 26 in communication with the guide wire lumen and a second connector 28 in fluid communication with the inflation lumen (not shown). The inflation lumen extends between balloon 20 and second connector 28. Both first and second connectors 26, 28 may optionally comprise standard connectors, such as Luer-Loc™ connectors.

Housing 29 also accommodates an electrical connector 38. Connector 38 includes a plurality of electrical connections, each electrically coupled to electrodes 34 via conductors (not shown). Electrodes 34 are energized and controlled by a controller 40 and power source 42, such as bipolar or monopolar RF energy, microwave energy, ultrasound energy, voltage source, current source, or other suitable energy source. In an embodiment, electrical connector 38 is coupled to an RF generator via a controller 40, with controller 40 allowing energy to be selectively directed to electrodes 34. When monopolar RF energy is employed, the patient may be grounded by connecting an external electrode, or an electrode connected to the catheter body 14, to the patient.

The controller 40 includes a processor, or is coupled to a processor, to control and/or record treatment. The processor will typically comprise computer hardware and/or software, often including one or more programmable processor units running machine readable program instructions or code for implementing some or all of one or more of the methods described herein. The code will often be embodied in a tangible media such as a memory (optionally a read only memory, a random access memory, a non-volatile memory, or the like) and/or a recording media (such as a floppy disk, a hard drive, a CD, a DVD, a non-volatile solid-state memory card, or the like). The code and/or associated data and signals may also be transmitted to or from the processor via a network connection, and some or all of the code may also be transmitted between components of catheter system 10 and within processor 40.

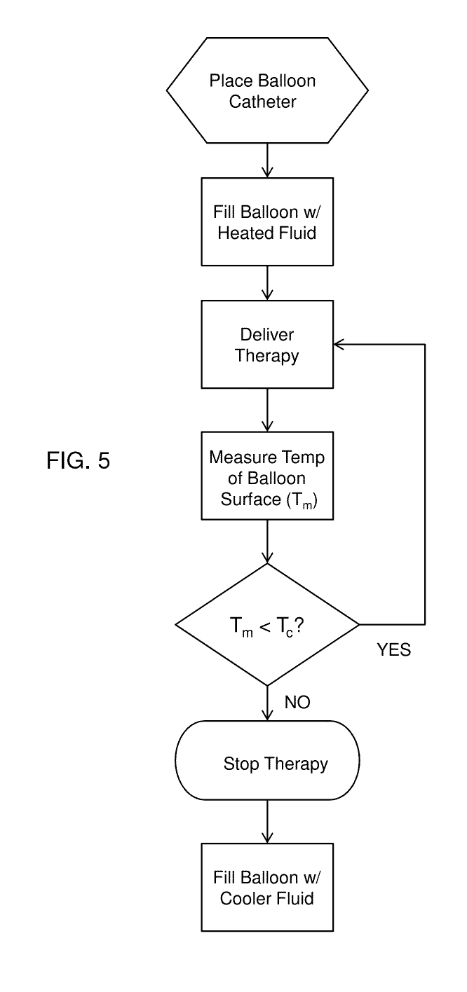

The balloon 20 generally includes a proximal portion coupled to an inflation lumen and a distal portion coupled to a guide wire lumen. (See FIGS. 4 and 5.) The balloon 20 expands radially when inflated with a fluid or a gas. In an embodiment, the balloon 20 is constructed from a compliant material that can withstand heat and high pressures. The balloon 20 may be constructed from polyethylene, nylon, polyvinylchloride, or polyethylene terephthalate. The balloon 20 typically is on the order of 2-7 French, i.e., approximately 1-3 mm, in diameter, when in an unexpanded state. Once expanded, the expanding disrupting element may be on the order of 3-8 mm depending upon the pressure on the expanding element and the compliance of the material. In some embodiments, the expanding element will be constructed from a high-compliance material that is able to withstand pressures on the order of 6 to 10 atm. Prior to inflation, the balloon 20 is positioned in the distal end 18 of the catheter. The balloon 20 may have helical folds to facilitate conversion between an expanded (inflated) configuration and a low profile configuration, needed for delivery and removal.

Catheter bodies intended for intravascular introduction will typically have a length in the range from 50 cm to 200 cm and an outer diameter in the range from 1 French to 12 French (0.33 mm: 1 French), usually from 3 French to 9 French. In the case of fistula treatment catheters, the length is typically in the range from 60 cm to 150 cm, the diameter is preferably below 8 French, more preferably below 7 French, and most preferably in the range from 2 French to 7 French.

Catheter bodies will typically be composed of a biocompatible polymer that is fabricated by conventional extrusion techniques. Suitable polymers include polyvinylchloride, polyurethanes, polyesters, polytetrafluoroethylenes (PTFE), silicone rubbers, natural rubbers, and the like. Optionally, the catheter body may be reinforced with braid, helical wires, coils, axial filaments, or the like, in order to increase rotational strength, column strength, toughness, pushability, and the like. Suitable catheter bodies may be formed by extrusion, with one or more channels being provided when desired. The catheter diameter can be modified by heat expansion and shrinkage using conventional techniques. The resulting catheters will thus be suitable for introduction to the vascular system, often the coronary arteries, by conventional techniques.

In an embodiment, the balloon 20 is configured with electrodes 34 integrated into the wall of the balloon 20 to deliver RF energy to heat tissues. The electrodes 34 may be mounted on an inside surface of balloon 20, with associated connectors/wires extending proximally from the electrodes. The electrodes 34 may be sandwiched between layers of balloon material. The electrodes 34 may be arranged in any suitable pattern, such as stripes, helixes, saw tooth, rings, or arrays.

The system may be used for monopolar or bipolar application of energy. For delivery of monopolar energy, a ground electrode is used, either on the catheter shaft, or on the patient's skin, such as a ground electrode pad. For delivery of bipolar energy, adjacent electrodes are axially offset to allow bipolar energy to be directed between adjacent circumferential (axially offset) electrodes. In other embodiments, electrodes may be arranged in bands around the balloon to allow bipolar energy to be directed between adjacent distal and proximal electrodes.

In another embodiment, the system heats tissues using heated fluids. In this configuration, balloon 20 need not include electrodes 34. In this embodiment, the balloon is substantially impervious to aqueous solutions, e.g., saline, to prevent the heated fluid from leaving the balloon. In an embodiment, the catheter includes an insulated lumen for delivering heated fluids to the balloon, e.g., heated saline. The fluid may have a temperature of 37° C. or greater, e.g., 40° C. or greater, e.g., 45° C. or greater, e.g., 50° C. or greater, e.g., 55° C. or greater, e.g., 60° C. or greater, e.g., 65° C. or greater, e.g., about 68° C. Systems of the catheter 10, configured to heat tissues with heated fluids may comprise a heated fluid reservoir and a pump connected to the inflation lumen to deliver the heated fluids (not shown). Other embodiments for heating tissues with heated fluids may comprise a heating element inside of the balloon as an element of the catheter. The balloon may be filled with room or body temperature saline directed to the balloon via an inflation lumen, and then the fluid can be heated with the heating element to provide a heated fluid. In some embodiments, a balloon catheter will also include a temperature sensor located proximate to the center of the balloon to be used to measure the temperature of the heated fluid.

In an embodiment, the balloon 20 is configured with temperature sensors integrated into the wall of the balloon. The temperature sensors may be mounted on an inside surface of balloon 20, with associated connectors/wires extending proximally from the temperature sensors. The temperature sensors may be mounted on an inside surface of the balloon 20. The temperature sensors may be sandwiched between layers of balloon material. The temperature sensors may be arranged in any suitable pattern, such as an array. The temperature sensors may be any temperature sensor that has a sufficiently small profile to be incorporated into the balloon, for example the temperature sensors may be a thermocouple, thermistor, thermal diode, or other suitable device. In some embodiments, the catheter will comprise an additional heating element that is inside the balloon, e.g., in proximity to a distal end of the inflation lumen, thereby allowing the inflation fluid, e.g., a heated inflation fluid, to be monitored.

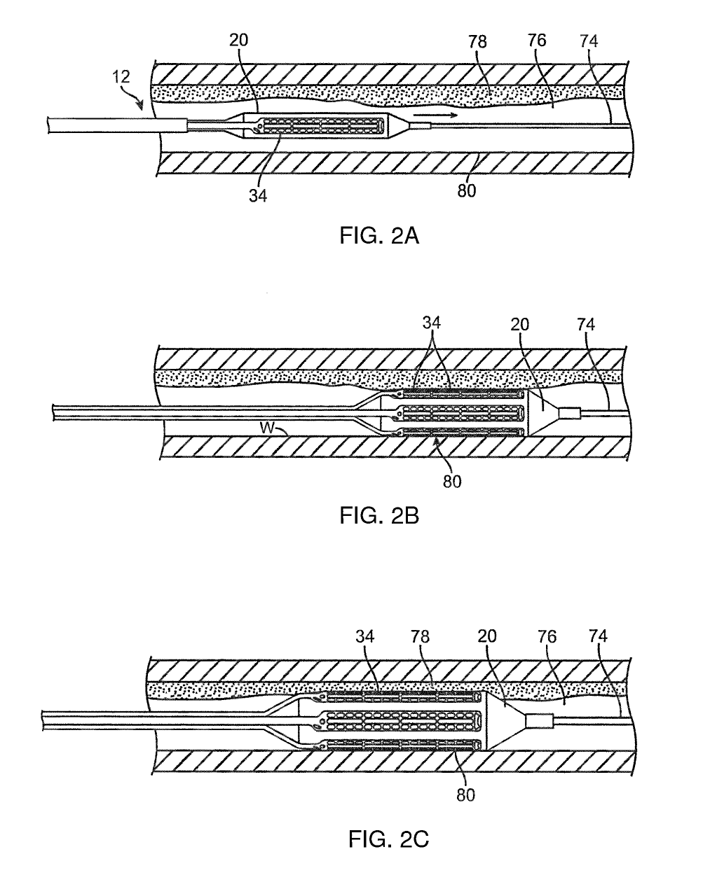

A generalized depiction of an ablation process is shown in FIGS. 2A-2C. FIGS. 2A-2C show resculpting of a vessel having a plaque deposit and/or thrombus, however the method is analogous to the method used to denerve the renal artery. As seen in FIG. 2A, accessing a treatment site will typically involve advancing a guide wire 74 within a blood vessel 76 to a targeted tissue, such as atherosclerotic material 78. Locating the balloon 20 may be facilitated by radiopaque markers or by radiopaque structures on or near the balloon 20. In some instances a guide wire suitable for use with an RF delivery system will be used, such as Safe-Cross™ RF system guide wire. The guide wire may also have imaging or measurement abilities such as the FLOWIRE® Doppler guide wire (Volcano Corporation, San Diego, Calif.). Typically the guide wire will be positioned under fluoroscopic (or other) imaging.

Regarding FIG. 2A, catheter 12 is advanced distally over guide wire 74 and positioned adjacent to the tissue to be treated, i.e., atherosclerotic material 78. As shown in FIG. 2B, the balloon 20 is expanded radially within the lumen of the blood vessel so that electrodes 34 radially engage atherosclerotic material 78. (In denerving an artery, the balloon is simply expanded to the vessel wall) In some instances, electrodes 34 will engage both atherosclerotic material 78 and healthy tissue 80.

Once the balloon 20 has engaged the vessel wall tissues, the electrodes 34 will be energized to treat the tissue. As shown in FIG. 2C, RF energy is directed to adjacent pairs of electrodes, treating both atherosclerotic material 78 and the healthy tissue 80. Most treatments are in the 1 to 6 Watt range, and are performed for a duration of 0.5 to 6 seconds. The duration and power are controlled using feedback from temperature sensors in the balloon, discussed in detail below. Using temperature sensors assures that the tissues are not overheated, but yet heated enough to affect the desired change in the tissue. In some embodiments, the power and duration may also be gated to assure that not enough energy is delivered to cause severe damage to the surrounding tissues.

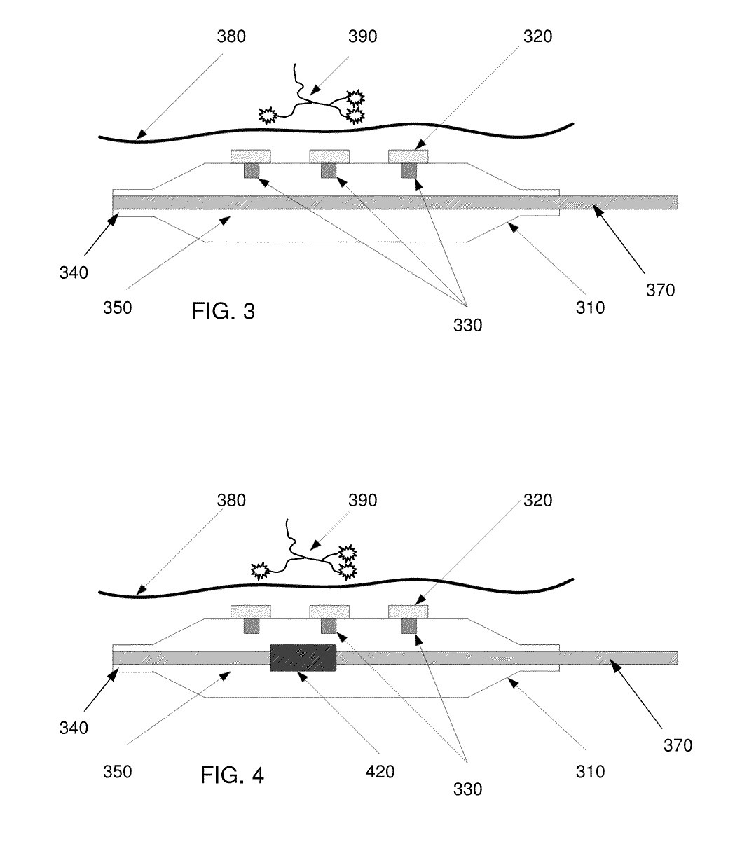

Catheters of the invention are described in greater detail in FIGS. 3 and 4. FIG. 3 shows an expandable member 310 in proximity to a tissue 380 to be treated. Heating elements 320 and temperature sensors 330 are integrated into expandable member 310. While the expandable member 310 is depicted as a balloon, alternative embodiments may have expandable members 310 that are not balloons. For example, the expandable member 310 could be constructed from a memory wire, such as nitinol, having suitably placed heating elements 320 and temperature sensors 330 to achieve heating of the tissues while monitoring the temperature of the tissue. As shown in FIG. 3, the expandable member 310 is delivered along a guide wire 370 and is connected to a lumen 340 that is a source for heated fluid. Once expanded, the inner volume 350 of the expandable member may be filled with a heated fluid, i.e., a fluid having a temperature greater than 60° C. In an alternative embodiment, shown in FIG. 4, the inner volume 350 is filled with a room temperature or body temperature fluid and then the fluid is heated with heating element 420. The fluid, i.e., the inflation fluid, is typically a biocompatible aqueous solution, such as saline or Ringer's solution. The fluid may additionally comprise contrast agents to facilitate visualization of the balloon and the status of the balloon, i.e., inflated or not inflated. The inner volume 350 can also be filled with a heated fluid and then further heated with heating element 420. In some embodiments, the catheter of FIG. 4 may also include an additional temperature sensor in proximity to the guide wire 370, capable of measuring the temperature of the heated fluid in the inner volume 350, but away from heating elements 330.

Once the expandable member 310 is expanded and filled with a heated fluid, the heating elements 320 will be energized to deliver energy to the tissue 380. Depending upon the procedure, the energy delivered to the tissue 380 will ablate the tissue 380 or affect a change to tissues/structures nearby the tissue 380 such as a nerve 390. Thus, in a renal denervation procedure, the nerve 390 will be disabled by the delivered energy. While the energy is delivered via heating elements 320, the temperature sensors 330 will monitor the temperature of the tissue to assure that the tissue does not exceed a safe temperature. The temperature sensors 330 will also monitor the temperature of the tissue to assure that it reaches the temperature needed for treatment.

In some embodiments, the optimum temperature to achieve renal denervation is 68° C. In this embodiment, the inner volume 350 is filled with a heated fluid also having a temperature of 68° C. As discussed previously, the heated fluid can be provided externally, e.g., through an insulated lumen, or the fluid can be heated once inside the expanding member, e.g., with heating element 420. Because the catheters are filled with a heated fluid that matches the desired tissue temperature, the heated solution cannot act as a heat sink against the heating elements 320, as is the case with current ablation catheters. Thus, the temperature sensors 330 will not sense a temperature that is lower than the actual temperature of the tissue, thereby assuring that the tissue is not overheated and damaged. Additionally, the heated fluid will help to provide even heating to the tissue to assure that the desired tissues do reach the desired temperatures.

When using an expandable member 310 configured as described above, it will not be necessary to gate the energy delivery during treatment. Rather, it will be a simple matter of comparing the temperature measured with the temperature sensor 330 to a predetermined temperature, e.g., 68° C. This method is shown in greater detail in FIG. 5. As discussed above with respect to FIG. 2A-C, the method begins with placing the expandable member 310 (e.g., balloon) near the tissue to be treated. The balloon is filled with a heated fluid and therapy is delivered by way of heating elements 320. Temperature sensors 330 monitor the temperature of the tissue being treated to determine a measured temperature (Tₘ). Tₘ is then compared to a predetermined temperature for treatment, T_{c}, or critical temperature. If Tₘ is less than T_{c}, the catheter is allowed to continue delivering therapy via the heating elements 320. However if Tₘ is equal to or greater than T_{c}, the heating elements are turned off, to avoid damaging the tissue. Once the therapy is completed, the heated fluid will be removed, typically by flushing away with a cooler fluid, e.g., room temperature saline. In other embodiments, the heated fluid may be simply evacuated with suction.

Advanced embodiments of the methods may include algorithms for monitoring or measuring the treatment area temperature. For example, readings from multiple temperature sensors 330 at different points on expandable member 330 may be modeled to develop a heat map of how the tissue is heating. Additionally, if the heating elements are individually addressable, it may be possible to turn some off and leave others on in order to achieve more even heating. Other algorithms may be used to estimate overshoot to determine if and when the heating elements should be turned off prior to Tₘ exceeding T_{c}.

In some embodiments, a catheter of the invention will additionally include imaging capabilities, such as intravascular ultrasound (IVUS) imaging or optical coherence tomography (OCT). The IVUS imaging assembly may be phased array IVUS imaging assembly, an pull-back type IVUS imaging assembly, or an IVUS imaging assembly that uses photoacoustic materials to produce diagnostic ultrasound and/or receive reflected ultrasound for diagnostics. IVUS imaging assemblies and processing of IVUS data are described for example in Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et at., U.S. Pat. No. 5,373,845, Seward et al., Mayo Clinic Proceedings 71(7):629-635 (1996), Packer et al., Cardiostim Conference 833 (1994), “Ultrasound Cardioscopy,” Eur. J.C.P.E. 4(2):193 (June 1994), Eberle et al., U.S. Pat. No. 5,453,575, Eberle et al., U.S. Pat. No. 5,368,037, Eberle et at., U.S. Pat. No. 5,183,048, Eberle et al., U.S. Pat. No. 5,167,233, Eberle et at., U.S. Pat. No. 4,917,097, Eberle et at., U.S. Pat. No. 5,135,486, and other references well known in the art relating to intraluminal ultrasound devices and modalities. All of these references are incorporated by reference herein.

In other embodiments, the imaging may use optical coherence tomography (OCT). OCT is a medical imaging methodology using a miniaturized near infrared light-emitting probe, and is capable of acquiring micrometer-resolution, three-dimensional images from within optical scattering media (e.g., biological tissue). OCT systems and methods are generally described in Castella et al., U.S. Pat. No. 8,108,030, Milner et al., U.S. Patent Application Publication No. 2011/0152771, Condit et al., U.S. Patent Application Publication No. 2010/0220334, Castella et al., U.S. Patent Application Publication No. 2009/0043191, Milner et al., U.S. Patent Application Publication No. 2008/0291463, and Kemp, N., U.S. Patent Application Publication No. 2008/0180683, the content of each of which is incorporated by reference in its entirety.

Other embodiments of catheters and methods of using them, not disclosed herein, will be evident to those of skill in the art, and are intended to be covered by the claims listed below.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A balloon catheter for ablating a tissue of a subject, comprising
an expandable balloon having a first temperature sensor integrated into a balloon wall;
a catheter body having a distal end and a proximal end and configured to deliver the expandable balloon to a tissue with the distal end of the catheter;
a lumen in fluid communication with the expandable balloon and the proximal end of the catheter; and
a second temperature sensor, located at the distal end of the catheter and configured to measure a temperature of a fluid within the balloon.

2. The balloon catheter of claim 1, wherein the balloon wall is substantially impervious to aqueous solutions.

3. The balloon catheter of claim 1, wherein the expandable balloon further comprises a heating element integrated into the balloon wall.

4. The balloon catheter of claim 3, wherein the heating element is a resistive heating element or an RF heating element.

5. The balloon catheter of claim 1, further comprising a heating element in contact with the lumen and configured to heat a fluid within the balloon.

6. The balloon catheter of claim 1, wherein the lumen is insulated.

7. The balloon catheter of claim 1, further comprising an imaging assembly.

8. The balloon catheter of claim 7, wherein the imaging assembly comprises an intravenous ultrasound (IVUS) imaging assembly or an optical coherence tomography (OCT) imaging assembly.

9. A method for applying energy to a tissue in a subject, comprising:
placing an expandable member having a temperature sensor in proximity to a tissue;
expanding the expandable member to cause the expandable member to contact the tissue;
providing a heated fluid to the expandable member;
applying energy to the tissue;
measuring a temperature of the tissue with the temperature sensor in the presence of the heated fluid; and
determining if it is safe to apply more energy to the tissue by comparing the measured temperature to a predetermined temperature.

10. The method of claim 9, wherein the expandable member is a balloon.

11. The method of claim 10, wherein the heated fluid is used to expand the balloon.

12. The method of claim 10, wherein the temperature sensor is integrated into a wall of the balloon.

13. The method of claim 10, wherein a heating element is integrated into a wall of the balloon.

14. The method of claim 13, wherein the heating element is a resistive heating element or an RF heating element.

15. The method of claim 10, wherein placing comprises delivering a catheter comprising the balloon in proximity to the tissue.

16. The method of claim 15, wherein the catheter additionally comprises a heating element for heating a fluid to provide a heated fluid to the balloon.

17. The method of claim 15, wherein the catheter additionally comprises an additional temperature sensor for measuring a temperature of the heated fluid.

18. The method of claim 9, wherein providing comprises delivering the heated fluid to the expandable member through an insulated lumen.

19. The method of claim 9, wherein the heated fluid is 60° C. or greater.

20. The method of claim 9, additionally comprising imaging the tissue.
